(19) United States
(12) Patent Application Publication
(10) Pub. No.: US 2023/0118152 A1
(43) Pub. Date: Apr. 20, 2023

(54) FORMS OF 5-({[2-AMINO-3-(4-CARBAMOYL-2,6-DIMETHYL-PHENYL)-PROPIONYL]-[1-(4-PHENYL-1H-IMIDAZOL-2-YL)-ETHYL]-AMINO}-METHYL)-2-METHOXY-BENZOIC ACID

(71) Applicant: Allergan Holdings Unlimited Company, Dublin (IE)

(72) Inventors: Thomas Karami, Aliso Viejo, CA (US); Qi Gao, Franklin Park, NJ (US)

(21) Appl. No.: 17/915,686

(22) PCT Filed: Mar. 30, 2021
(86) PCT No.: PCT/IB21/00209

§ 371 Date: Sep. 29, 2022

Related U.S. Application Data
(60) Provisional application No. 63/001,969, filed on Mar. 30, 2020.

(51) Int. Cl. A61K 31/417

(52) U.S. Cl. CPC A61K 31/417

(57) ABSTRACT

The present invention relates to novel crystalline Forms C, C′, D′ and H3 of 5-({[2-amino (4-carbamoyl-2,6-dimethyl-phenyl)-propionyl]-[1-(4-phenyl-1H-imidazol-2-yl)-ethyl]-amino}-methyl)-2-methoxy-benzoic acid and methods of preparing the same.

### RELATED APPLICATIONS

This application claims the benefit of priority to U.S. Provisional Patent Application No. 63/001,969, filed Mar. 30, 2020. This application is incorporated herein by reference in its entirety.

### FIELD OF THE INVENTION

The present invention relates to novel crystalline forms of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid, methods of making them and uses thereof in the preparation or production of pharmaceutical drug dosage forms.

### BACKGROUND OF THE INVENTION

The delivery of an active pharmaceutical ingredient (“API”) to a patient requires more than simply the identification of a molecule and its methods of use. An API must be formulated for delivery to a patient and such formulation (in addition to the API activity) is evaluated by regulatory agencies such as the US Food and Drug Administration (FDA) and the European Medicines Agency (EMEA). The API's formulation affects, among others, delivery profile, stability, consistency, and manufacturing controls. An important factor in determining the properties of a formulation is the form of the API. APIs have been known to exist as amorphous forms, crystalline forms, hydrates and solvates, so called polymorphs. While one API may be known to have one or multiple polymorphs (e.g. crystalline anhydrous, hydrated- or solvated forms in addition to its amorphous form), another API may be known to only exist in amorphous form. The form diversity is important because each different polymorphic form, (i.e. anhydrous, crystalline solvate(s), crystalline hydrate(s) or amorphous form) may have different physicochemical properties such as stability, solubility, intrinsic dissolution rate, melting temperature and hygroscopicity.

Some forms of an API can be formulated into a pharmaceutical formulation suitable for human use, while other forms lack the required properties for such uses. Even if an API can exist in more than one form suitable for formulation, different properties of an API form can affect the manufacturing process, shelf-life, route of administration, bioavailability and other important product characteristics. For example, the ability to improve or modulate stability or hygroscopicity can decrease manufacturing costs by reducing the need for humidity-controlled chambers or reducing the need to package an API in humidity resistant packaging. In addition, these same changes can increase product shelf stability thereby improving product distribution possibilities and affecting cost. In another example, one polymorphic form of an API may have greater bioavailability than another form. Choosing a form that provides the higher bioavailability allows for a lower drug dose to be administered to a patient. Selecting the most stable hydrate of the drug for development of aqueous based oral suspension formulation(s) provides a better physical stability of the drug in oral suspension, micro-suspension and nano-suspension products, e.g., pediatric oral suspensions, micro-suspensions and nano-suspensions. A metastable solvate such as isostructural methanolate, tri-methanolate, isopropanol solvates, etc. can be used as an intermediate form to manufacture a more stable form, or the thermodynamically most stable form of an API.

Further, changes to the process of making an API can result in less processing steps, higher purity and lower cost. Such advantages are important to the pharmaceutical industry.

5-({[2-Amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, also known as eluxadoline (structure shown in FIG. 5), is an opioid receptor modulator (mu opioid receptor agonist and delta opioid receptor antagonist) used for treating irritable bowel syndrome, pain or other opioid receptor disorders. 5-({[2-Amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid and methods of making this molecule are disclosed in US application 2005/02033143. Example 9 of US application 2005/02033143 makes the hydrochloride salt of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. US application 2009/0018179 describes a zwitterion of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid and two crystalline forms α and β of this compound. Crystalline forms α and β provided improved properties over the amorphous form and can be purified at higher purity. The α and β crystals may be interchangeably referred to herein as “Form A” and “Form B” crystals, respectively. The entire contents of US application 2005/02033143 and 2009/0018179 are incorporated herein by reference. Other polymeric forms of eluxadoline are described in PCT/US2016/043678, published as WO 2017/015606, and PCT/IB2019/056988.

Additional polymeric forms of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid having different manufacturing and formulation applications or improved properties are needed in the art and are described herein.

### SUMMARY OF THE INVENTION

The present invention relates to novel crystalline Forms C (isostructural solvate), C′ (tri-methanolate), D′ (monohydrate) and H3 (trihydrate) of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. The invention also provides pharmaceutical compositions comprising these novel crystalline forms. Compositions and methods of the invention are useful in the treatment or prevention of a variety of diseases including, among others, irritable bowel syndrome, pain and other opioid receptor mediated disorders.

In one embodiment, the invention relates to a novel Form C crystal of eluxadoline characterized by a powder X-ray diffraction pattern comprising the powder X-ray diffraction peaks at 2-theta values of 7.2±0.2, 11.8±0.2, 12.1±0.2, 12.7±0.2, 15.0±0.2, 15.8±0.2, 16.1±0.2, 18.2±0.2, 19.2±0.2, 19.9±0.2, 22.6±0.2, and 25.0±0.2 degrees.

In another embodiment, the Form C crystal may be characterized by at least a minimum corresponding number of powder X-ray diffraction peaks at 7.2±0.2, 11.8±0.2, 12.1±0.2, 12.7±0.2, 15.0±0.2, 15.8±0.2, 16.1±0.2, 18.2±0.2, 19.2±0.2, 19.9±0.2, 22.6±0.2, and 25.0±0.2 degrees, wherein the minimum corresponding number is three, four, five, six, seven, eight, nine, ten or more than ten.

In some embodiments, the Form C crystal may be characterized by a powder X-ray diffraction pattern having powder X-ray diffraction peaks at about 7.2±0.2, 12.1±0.2, and 19.2±0.2 degrees 2-theta. In some embodiments, the Form C crystal may be characterized by a powder X-ray diffraction pattern having powder X-ray diffraction peaks at about 7.2±0.2, 12.1±0.2, 19.2±0.2 and 11.8±0.2 degrees 2-theta. In some embodiments, the Form C crystal may be characterized by a powder X-ray diffraction pattern having powder X-ray diffraction peaks at about 7.2±0.2, 12.1±0.2, 19.2±0.2, 11.8±0.2 and 15.0±0.2 degrees 2-theta.

In some embodiments, the Form C′ crystal (tri-methanolate) may be characterized by a powder X-ray diffraction pattern comprising the powder X-ray diffraction peaks at 2-theta values of about 6.6±0.2, 6.7±0.2, 8.8±0.2, 10.4±0.2, 11.4±0.2, 11.8±0.2, 11.9±0.2, 13.2±0.2, 14.5±0.2, 17.6±0.2, 18.1±0.2, 20.2±0.2, 21.0±0.2, 21.7±0.2, 22.6±0.2, 23.2±0.2, 24.7±0.2 and 26.6±0.2 degrees.

In another embodiment, the Form C′ crystal may be characterized by at least a minimum corresponding number of powder X-ray diffraction peaks of 2-theta values of about 6.6±0.2, 6.7±0.2, 8.8±0.2, 10.4±0.2, 11.4±0.2, 11.8±0.2, 11.9±0.2, 13.2±0.2, 14.5±0.2, 17.6±0.2, 18.1±0.2, 20.2±0.2, 21.0±0.2, 21.7±0.2, 22.6±0.2, 23.2±0.2, 24.7±0.2 and 26.6±0.2 degrees, wherein the minimum corresponding number of X-ray diffraction peaks is three, four, five, six, seven, eight, nine, ten or more than ten.

In some embodiments, the Form C′ crystal may be characterized by a powder X-ray diffraction pattern having powder X-ray diffraction peaks at about 6.7±0.2, 21.0±0.2 and 6.6±0.2 degrees 2-theta. In some embodiments, the Form C′ crystal may be characterized by a powder X-ray diffraction pattern having powder X-ray diffraction peaks at about 6.7±0.2, 21.0±0.2, 6.6±0.2 and 10.4±0.2 degrees 2-theta. In some embodiments, the Form C′ crystal may be characterized by a powder X-ray diffraction pattern having powder X-ray diffraction peaks at about 6.7±0.2, 21.0±0.2, 6.6±0.2, 10.4±0.2 and 11.8±0.2 degrees 2-theta.

In some embodiments, the Form D′ crystal (monohydrate) may be characterized by a powder X-ray diffraction pattern comprising any three or more powder X-ray diffraction peaks at 2-theta values of about 8.9±0.2, 9.2±0.2, 11.1±0.2, 11.3±0.2, 12.0±0.2, 13.7±0.2, 16.0±0.2, 17.4±0.2, 17.8±0.2, 17.9±0.2, 20.5±0.2, 20.8±0.2, 21.3±0.2, 21.7±0.2, 21.8±0.2, 22.0±0.2, 25.0±0.2, 26.8±0.2, 27.6±0.2 and 29.1±0.2 degrees.

In another embodiment, the Form D′ crystal may be characterized by at least a minimum corresponding number of powder X-ray diffraction peaks of 2-theta values of about 8.9±0.2, 9.2±0.2, 11.1±0.2, 11.3±0.2, 12.0±0.2, 13.7±0.2, 16.0±0.2, 17.4±0.2, 17.8±0.2, 17.9±0.2, 20.5±0.2, 20.8±0.2, 21.3±0.2, 21.7±0.2, 21.8±0.2, 22.0±0.2, 25.0±0.2, 26.8±0.2, 27.6±0.2 and 29.1±0.2 degrees, wherein the minimum corresponding number of X-ray diffraction peaks is three, four, five, six, seven, eight, nine, ten or more than ten.

In some embodiments, the Form D′ crystal may be characterized by a powder X-ray diffraction pattern having powder X-ray diffraction peaks at about 16.0±0.2, 12.0±0.2 and 11.1±0.2 degrees 2-theta. In some embodiments, the Form D′ crystal may be characterized by a powder X-ray diffraction pattern having powder X-ray diffraction peaks at about 16.0±0.2, 12.0±0.2, 11.1±0.2 and 8.9±0.2 degrees 2-theta. In some embodiments, the Form D′ crystal may be characterized by a powder X-ray diffraction pattern having powder X-ray diffraction peaks at about 16.0±0.2, 12.0±0.2, 11.1±0.2, 8.9±0.2 and 27.6±0.2 degrees 2-theta.

In some embodiments, the Form H3 crystal (trihydrate) may be characterized by a powder X-ray diffraction pattern comprising any three or more powder X-ray diffraction peaks at 2-theta values of about 8.1±0.2, 11.0±0.2, 12.4±0.2, 13.2±0.2, 14.8±0.2, 15.2±0.2, 16.6±0.2, 17.9±0.2, 18.7±0.2, 18.9±0.2, 19.1±0.2, 20.0±0.2 and 24.3±0.2 degrees.

In another embodiment, the Form H3 crystal may be characterized by at least a minimum corresponding number of powder X-ray diffraction peaks of 2-theta values of about 8.1±0.2, 11.0±0.2, 12.4±0.2, 13.2±0.2, 14.8±0.2, 15.2±0.2, 16.6±0.2, 17.9±0.2, 18.7±0.2, 18.9±0.2, 19.1±0.2, 20.0±0.2 and 24.3±0.2 degrees, wherein the minimum corresponding number of X-ray diffraction peaks is three, four, five, six, seven, eight, nine, ten or more than ten.

In some embodiments, the Form H3 crystal may be characterized by a powder X-ray diffraction pattern having powder X-ray diffraction peaks at about 8.1±0.2, 14.8±0.2 and 16.6±0.2 degrees 2-theta. In some embodiments, the Form H3 crystal may be characterized by a powder X-ray diffraction pattern having powder X-ray diffraction peaks at about 8.1±0.2, 14.8±0.2, 16.6±0.2 and 17.9±0.2 degrees 2-theta. In some embodiments, the Form H3 crystal may be characterized by a powder X-ray diffraction pattern having powder X-ray diffraction peaks at about 8.1±0.2, 14.8±0.2, 16.6±0.2, 17.9±0.2 and 19.1±0.2 degrees 2-theta.

### BRIEF DESCRIPTION OF THE DRAWINGS

FIG. 1 illustrates X-ray powder diffraction (XRPD) measurements of a representative crystalline Form C.

FIG. 2 illustrates powder X-ray diffraction (PXRD) measurements of a representative crystalline Form C′.

FIG. 3 illustrates powder X-ray diffraction (PXRD) measurements of a representative crystalline Form D′.

FIG. 4 illustrates powder X-ray diffraction (PXRD) measurements of a representative crystalline Form H3.

FIG. 5 illustrates the structure of eluxadoline.

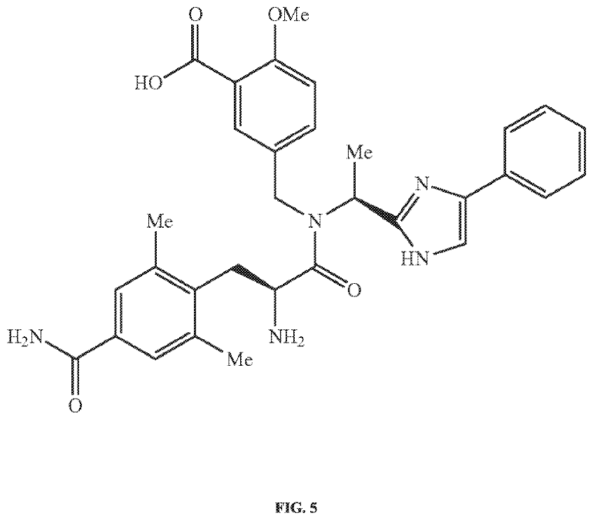

### DETAILED DESCRIPTION

The present invention includes novel crystalline forms of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In one aspect of the invention, the invention includes a novel Form C crystal (isostructural solvate) of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. Form C may be useful for treating pain, irritable bowel syndrome, or other opioid receptor disorders in mammals, by administering to said mammal an effective amount of a Form C crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the Form C crystal may be characterized by a powder X-ray diffraction pattern comprising the powder X-ray diffraction peaks at 2-theta values of 7.2±0.2, 11.8±0.2, 12.1±0.2, 12.7±0.2, 15.0±0.2, 15.8±0.2, 16.1±0.2, 18.2±0.2, 19.2±0.2, 19.9±0.2, 22.6±0.2, and 25.0±0.2 degrees.

In another embodiment, the Form C crystal may be characterized by at least a minimum corresponding number of powder X-ray diffraction peaks at 2-theta values of about 7.2±0.2, 11.8±0.2, 12.1±0.2, 12.7±0.2, 15.0±0.2, 15.8±0.2, 16.1±0.2, 18.2±0.2, 19.2±0.2, 19.9±0.2, 22.6±0.2, and 25.0±0.2 degrees, wherein the minimum corresponding number is three, four, five, six, seven, eight, nine, ten or more than ten.

In some embodiments, the Form C crystal may be characterized by a powder X-ray diffraction pattern having powder X-ray diffraction peaks at about 7.2±0.2, 12.1±0.2, and 19.2±0.2 degrees 2-theta. In some embodiments, the Form C crystal may be characterized by a powder X-ray diffraction pattern having powder X-ray diffraction peaks at about 7.2±0.2, 12.1±0.2, 19.2±0.2 and 11.8±0.2 degrees 2-theta. In some embodiments, the Form C crystal may be characterized by a powder X-ray diffraction pattern having powder X-ray diffraction peaks at about 7.2±0.2, 12.1±0.2, 19.2±0.2, 11.8±0.2 and 15.0±0.2 degrees 2-theta.

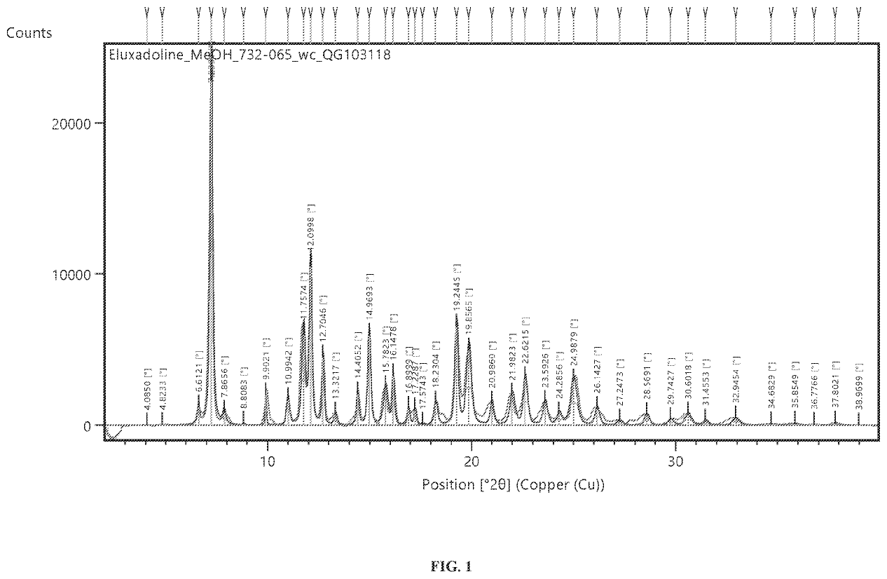

In some embodiments, the Form C crystal may be characterized by a powder X-ray diffraction pattern having powder X-ray diffraction peaks substantially as shown in Table 1. In some embodiments, the Form C crystal may be characterized by a powder X-ray diffraction pattern that may be substantially similar to the powder X-ray diffraction pattern of FIG. 1. In some embodiments, the Form C crystal may be substantially pure.

TABLE 1

| Position | Relative Intensity |
|---|---|
| [°2θ] | [%] |
| 6.6121 | 5.27 |
| 7.2391 | 100.00 |
| 9.9021 | 8.85 |
| 10.9942 | 7.50 |
| 11.7574 | 26.38 |
| 12.0998 | 46.12 |
| 12.7046 | 19.73 |
| 14.4052 | 9.08 |
| 14.9693 | 26.15 |
| 15.7823 | 10.87 |
| 16.1478 | 14.32 |
| 18.2304 | 6.48 |
| 19.2445 | 28.78 |
| 19.8565 | 21.91 |
| 20.9860 | 6.51 |
| 21.9823 | 8.77 |
| 22.6215 | 13.59 |
| 23.5926 | 6.67 |
| 24.9879 | 12.82 |
| 26.1427 | 5.00 |

In another aspect of the invention, the present invention also includes a novel Form C′ (tri-methanolate) crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. Form C′ may be useful for treating pain, irritable bowel syndrome, or other opioid receptor disorders in mammals, by administering to said mammal an effective amount of a Form C′ crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the Form C′ crystal may be characterized by a powder X-ray diffraction pattern comprising the powder X-ray diffraction peaks at 2-theta values of about 6.6±0.2, 6.7±0.2, 8.8±0.2, 10.4±0.2, 11.4±0.2, 11.8±0.2, 11.9±0.2, 13.2±0.2, 14.5±0.2, 17.6±0.2, 18.1±0.2, 20.2±0.2, 21.0±0.2, 21.7±0.2, 22.6±0.2, 23.2±0.2, 24.7±0.2 and 26.6±0.2 degrees.

In another embodiment, the Form C′ crystal may be characterized by at least a minimum corresponding number of powder X-ray diffraction peaks of 2-theta values of about 6.6±0.2, 6.7±0.2, 8.8±0.2, 10.4±0.2, 11.4±0.2, 11.8±0.2, 11.9±0.2, 13.2±0.2, 14.5±0.2, 17.6±0.2, 18.1±0.2, 20.2±0.2, 21.0±0.2, 21.7±0.2, 22.6±0.2, 23.2±0.2, 24.7±0.2 and 26.6±0.2 degrees, wherein the minimum corresponding number of powder X-ray diffraction peaks is three, four, five, six, seven, eight, nine, ten or more than ten.

In some embodiments, the Form C′ crystal may be characterized by a powder X-ray diffraction pattern having powder X-ray diffraction peaks at about 6.7±0.2, 21.0±0.2 and 6.6±0.2 degrees 2-theta. In some embodiments, the Form C′ crystal may be characterized by a powder X-ray diffraction pattern having powder X-ray diffraction peaks at about 6.7±0.2, 21.0±0.2, 6.6±0.2 and 10.4±0.2 degrees 2-theta. In some embodiments, the Form C′ crystal may be characterized by a powder X-ray diffraction pattern having powder X-ray diffraction peaks at about 6.7±0.2, 21.0±0.2, 6.6±0.2, 10.4±0.2 and 11.8±0.2 degrees 2-theta.

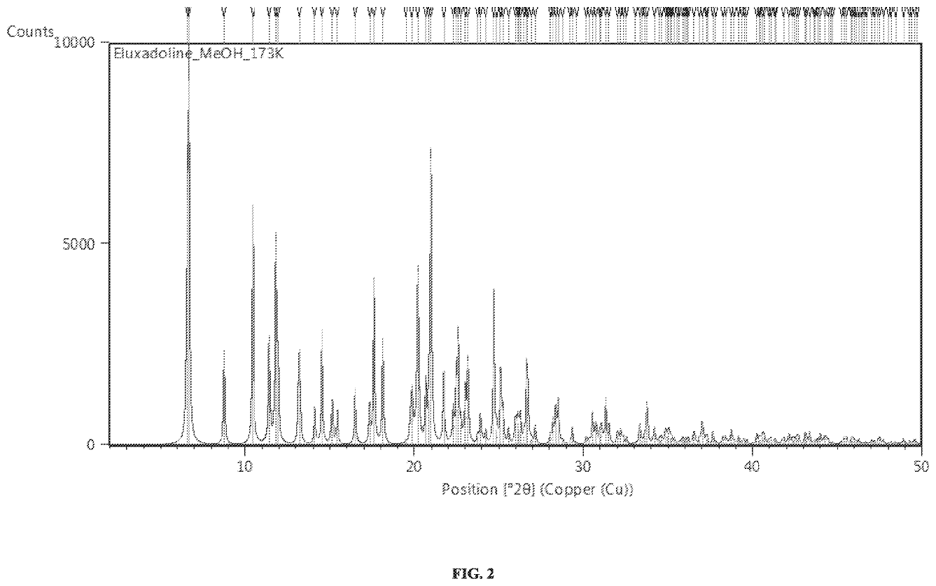

In some embodiments, the Form C′ crystal may be characterized by a powder X-ray diffraction pattern having powder X-ray diffraction peaks substantially as shown in Table 2. In some embodiments, the Form C′ crystal may be characterized by a powder X-ray diffraction pattern that may be substantially similar to the powder X-ray diffraction pattern of FIG. 2. In some embodiments, the Form C′ crystal may be substantially pure.

TABLE 2

| Position | Rel. Int. |
|---|---|
| [°2θ] | [%] |
| 6.5700 | 66.92 |
| 6.6790 | 100.00 |
| 8.7566 | 26.30 |
| 10.4482 | 65.54 |
| 11.4104 | 28.51 |
| 11.8135 | 55.54 |
| 11.9404 | 20.86 |
| 13.1929 | 26.04 |
| 14.1060 | 9.79 |
| 14.5368 | 31.39 |
| 15.1403 | 11.78 |
| 15.4342 | 9.20 |
| 16.4951 | 15.60 |
| 17.3547 | 10.34 |
| 17.6170 | 45.18 |
| 18.1277 | 29.32 |
| 19.8416 | 13.40 |
| 20.1972 | 47.60 |
| 20.6744 | 14.69 |
| 20.8565 | 12.03 |
| 20.9813 | 78.43 |
| 21.7424 | 19.87 |
| 22.3032 | 9.43 |
| 22.4770 | 18.10 |
| 22.5976 | 28.79 |
| 22.7633 | 5.89 |
| 23.0194 | 14.29 |
| 23.1737 | 22.81 |
| 23.9153 | 8.26 |
| 24.7078 | 42.71 |
| 25.1044 | 19.46 |
| 25.2354 | 9.36 |
| 25.9899 | 6.10 |
| 26.0974 | 5.89 |
| 26.2804 | 6.97 |
| 26.6403 | 20.22 |
| 26.7252 | 12.74 |
| 27.1455 | 5.07 |
| 28.1923 | 6.57 |
| 28.3614 | 9.57 |
| 28.5282 | 12.32 |
| 30.5510 | 8.44 |
| 31.3115 | 12.23 |
| 31.5197 | 5.00 |
| 33.3236 | 5.46 |
| 33.7650 | 11.17 |
| 37.0221 | 5.73 |

In yet another aspect of the invention, the present invention also includes a novel Form D′ (monohydrate) crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. Form D′ may be useful for treating pain, irritable bowel syndrome, or other opioid receptor disorders, by administering to said mammal an effective amount of a Form D′ crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the Form D′ crystal may be characterized by a powder X-ray diffraction pattern comprising any three or more powder X-ray diffraction peaks at 2-theta values of about 8.9±0.2, 9.2±0.2, 11.1±0.2, 11.3±0.2, 12.0±0.2, 13.7±0.2, 16.0±0.2, 17.4±0.2, 17.8±0.2, 17.9±0.2, 20.5±0.2, 20.8±0.2, 21.3±0.2, 21.7±0.2, 21.8±0.2, 22.0±0.2, 25.0±0.2, 26.8±0.2, 27.6±0.2 and 29.1±0.2 degrees.

In another embodiment, the Form D′ crystal may be characterized by at least a minimum corresponding number of powder X-ray diffraction peaks of 2-theta values of about 8.9±0.2, 9.2±0.2, 11.1±0.2, 11.3±0.2, 12.0±0.2, 13.7±0.2, 16.0±0.2, 17.4±0.2, 17.8±0.2, 17.9±0.2, 20.5±0.2, 20.8±0.2, 21.3±0.2, 21.7±0.2, 21.8±0.2, 22.0±0.2, 25.0±0.2, 26.8±0.2, 27.6±0.2 and 29.1±0.2 degrees, wherein the minimum corresponding number of powder X-ray diffraction peaks is three, four, five, six, seven, eight, nine, ten or more than ten.

In some embodiments, the Form D′ crystal may be characterized by a powder X-ray diffraction pattern having powder X-ray diffraction peaks at about 16.0±0.2, 12.0±0.2 and 11.1±0.2 degrees 2-theta. In some embodiments, the Form D′ crystal may be characterized by a powder X-ray diffraction pattern having powder X-ray diffraction peaks at about 16.0±0.2, 12.0±0.2, 11.1±0.2 and 8.9±0.2 degrees 2-theta. In some embodiments, the Form D′ crystal may be characterized by a powder X-ray diffraction pattern having powder X-ray diffraction peaks at about 16.0±0.2, 12.0±0.2, 11.1±0.2, 8.9±0.2 and 27.6±0.2 degrees 2-theta.

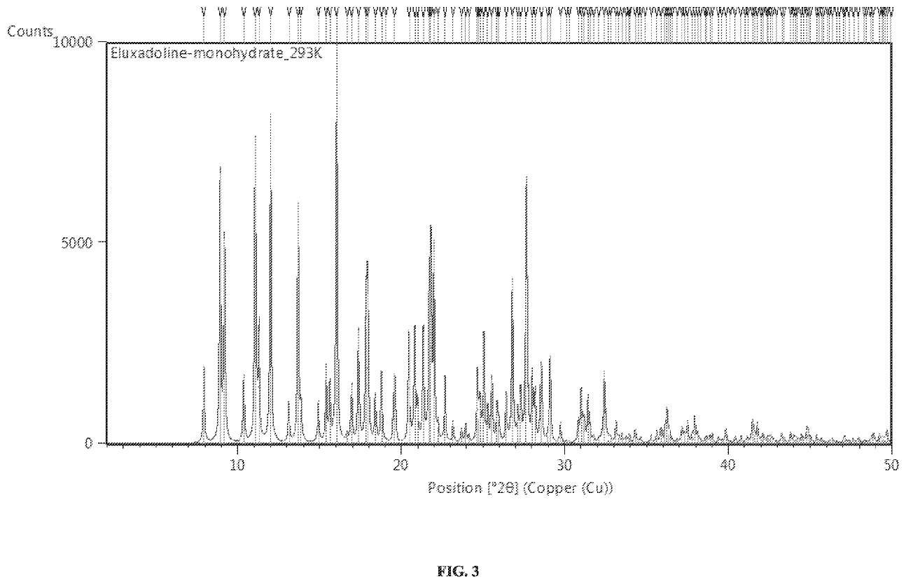

In some embodiments, the Form D′ crystal may be characterized by a powder X-ray diffraction pattern having powder X-ray diffraction peaks substantially as shown in Table 3. In some embodiments, the Form D′ crystal may be characterized by a powder X-ray diffraction pattern that may be substantially similar to the powder X-ray diffraction pattern of FIG. 3. In some embodiments, the Form D′ crystal may be substantially pure.

TABLE 3

| Position | Rel. Int. |
|---|---|
| [°2θ] | [%] |
| 7.9256 | 19.79 |
| 8.9169 | 67.64 |
| 9.1785 | 50.97 |
| 10.3629 | 16.74 |
| 11.0732 | 76.73 |
| 11.2857 | 28.13 |
| 12.0047 | 82.78 |
| 13.1259 | 10.14 |
| 13.6877 | 60.64 |
| 13.8674 | 8.40 |
| 14.9221 | 10.52 |
| 15.4011 | 18.62 |
| 15.6293 | 14.55 |
| 16.0409 | 100.00 |
| 16.9637 | 14.40 |
| 17.3813 | 28.18 |
| 17.8421 | 29.16 |
| 17.9396 | 37.51 |
| 18.4170 | 11.67 |
| 18.7942 | 17.92 |
| 19.5854 | 17.23 |
| 20.4631 | 27.42 |
| 20.8125 | 28.63 |
| 20.9858 | 9.17 |
| 21.3387 | 27.83 |
| 21.7003 | 37.89 |
| 21.8115 | 47.76 |
| 22.0062 | 47.85 |
| 22.6827 | 16.37 |
| 23.1697 | 5.51 |
| 24.6410 | 16.64 |
| 24.7492 | 6.05 |
| 24.8308 | 8.97 |
| 25.0426 | 26.65 |
| 25.2791 | 8.24 |
| 25.5395 | 16.06 |
| 25.8590 | 8.76 |
| 25.9654 | 5.84 |
| 26.4273 | 12.22 |
| 26.8013 | 40.89 |
| 27.1277 | 7.29 |
| 27.3070 | 12.42 |
| 27.6555 | 64.26 |
| 28.0231 | 15.88 |
| 28.1823 | 12.20 |
| 28.5612 | 19.59 |
| 29.1123 | 21.67 |
| 30.8391 | 5.51 |
| 31.0102 | 12.95 |
| 31.1739 | 5.92 |
| 31.4217 | 11.35 |
| 32.4212 | 17.15 |
| 33.1295 | 5.40 |
| 36.2618 | 7.56 |
| 37.5034 | 5.21 |
| 37.9482 | 6.56 |
| 41.4894 | 5.48 |

In yet another aspect of the invention, the present invention also includes a novel Form H3 (trihydrate) crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid. Form H3 may be useful for treating pain, irritable bowel syndrome, or other opioid receptor disorders, by administering to said mammal an effective amount of a Form H3 crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the Form H3 crystal may be characterized by a powder X-ray diffraction pattern comprising any three or more powder X-ray diffraction peaks at 2-theta values of about 8.1±0.2, 11.0±0.2, 12.4±0.2, 13.2±0.2, 14.8±0.2, 15.2±0.2, 16.6±0.2, 17.9±0.2, 18.7±0.2, 18.9±0.2, 19.1±0.2, 20.0±0.2 and 24.3±0.2 degrees.

In another embodiment, the Form H3 crystal may be characterized by at least a minimum corresponding number of powder X-ray diffraction peaks of 2-theta values of about 8.1±0.2, 11.0±0.2, 12.4±0.2, 13.2±0.2, 14.8±0.2, 15.2±0.2, 16.6±0.2, 17.9±0.2, 18.7±0.2, 18.9±0.2, 19.1±0.2, 20.0±0.2 and 24.3±0.2 degrees, wherein the minimum corresponding number of powder X-ray diffraction peaks is three, four, five, six, seven, eight, nine, ten or more than ten.

In some embodiments, the Form H3 crystal may be characterized by a powder X-ray diffraction pattern having powder X-ray diffraction peaks at about 8.1±0.2, 14.8±0.2 and 16.6±0.2 degrees 2-theta. In some embodiments, the Form H3 crystal may be characterized by a powder X-ray diffraction pattern having powder X-ray diffraction peaks at about 8.1±0.2, 14.8±0.2, 16.6±0.2 and 17.9±0.2 degrees 2-theta. In some embodiments, the Form H3 crystal may be characterized by a powder X-ray diffraction pattern having powder X-ray diffraction peaks at about 8.1±0.2, 14.8±0.2, 16.6±0.2, 17.9±0.2 and 19.1±0.2 degrees 2-theta.

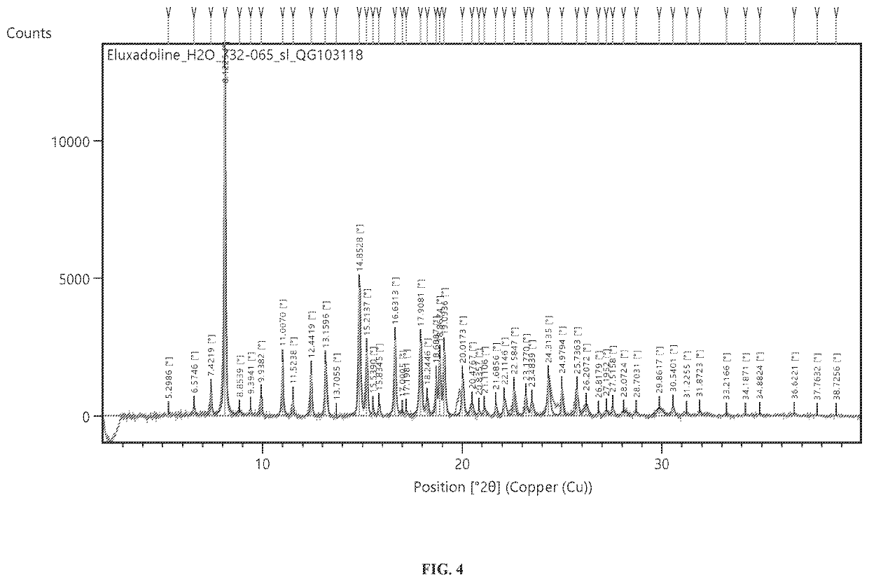

In some embodiments, the Form H3 crystal may be characterized by a powder X-ray diffraction pattern having powder X-ray diffraction peaks substantially as shown in Table 4. In some embodiments, the Form H3 crystal may be characterized by a powder X-ray diffraction pattern that may be substantially similar to the powder X-ray diffraction pattern of FIG. 4. In some embodiments, the Form H3 crystal may be substantially pure.

TABLE 4

| Position | Relative Intensity |
|---|---|
| [°2θ] | [%] |
| 7.4219 | 7.01 |
| 8.1225 | 100.00 |
| 9.9382 | 5.37 |
| 11.0070 | 15.70 |
| 12.4419 | 12.21 |
| 13.1596 | 15.22 |
| 14.8528 | 37.20 |
| 15.2137 | 18.72 |
| 16.6313 | 21.91 |
| 17.9081 | 21.46 |
| 18.6887 | 10.91 |
| 18.8674 | 16.77 |
| 19.0936 | 18.97 |
| 20.0173 | 10.86 |
| 22.5847 | 7.58 |
| 23.1770 | 5.84 |
| 24.3135 | 11.01 |
| 24.9794 | 7.97 |
| 25.7363 | 7.83 |

The X-ray powder diffraction data of each of the above crystalline forms were obtained by using X-ray source as Cu, (Kα radiation, λ=1.54 Å).

Pharmaceutical dosage forms of crystals of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid can be administered in several ways including, but not limited to, oral administration. Oral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Liquid dosage forms may also be provided by the present invention, including such non-limiting examples as a suspension, a solution, syrup, or an emulsion. In another embodiment, the present invention includes the preparation of a medicament comprising a crystalline or polymorphic form of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid. A Form E crystal of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid can be administered by controlled- or delayed-release means.

Like the amounts and types of excipients, the amounts and specific type of active ingredient in a dosage form may differ depending on factors such as, but not limited to, the route by which it is to be administered to mammals. However, typical dosage forms of the invention comprise a Form C, C′, D′ or H3 crystal of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid, in an amount of from about 0.10 mg to about 1 g, from about 0.2 mg to about 500 mg, or from about 1 mg to about 250 mg. Non-limiting examples include 0.2 mg, 0.5 mg, 0.75 mg, 1.0 mg, 1.2 mg, 1.5 mg, 2 mg, 3 mg, 5 mg, 7 mg, 10 mg, 25 mg, 50 mg, 75 mg, 100 mg, 150 mg, 200 mg, 250 mg, and 500 mg dosages. In some embodiments, the dosage forms comprise 75 mg or 100 mg dosages. The dosages, however, may be varied depending upon the requirement of the patients, the severity of the condition being treated and the compound being employed. The use of either daily administration or post-periodic dosing may be employed.

The crystals of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid of the present invention may also be used to prepare pharmaceutical dosage forms other than the oral dosage forms described above, such as topical dosage forms, parenteral dosage forms, transdermal dosage forms, and mucosal dosage forms. For example, such forms include creams, lotions, solutions, suspensions, emulsions, ointments, powders, patches, suppositories, and the like.

The crystals of 5-({[2-amino-3-(4-carbamoyl-2,6-dimethyl-phenyl)-propionyl]-[1-(4-phenyl-1h-imidazol-2-yl)-ethyl]-amino}-methyl)-2-methoxy-benzoic acid of the present invention can be characterized by any one, any two, any three, any four, any five, any six, any seven, any eight, any nine, or any ten PXRD 2-theta angle peaks, or by any combination of the data acquired from the analytical techniques described above which distinctly identify the particular crystal.

In an embodiment, a pharmaceutical composition of this invention also may include combinations of the different crystalline forms of eluxadoline described herein, amorphous eluxadoline, crystalline Forms α and β as described in U.S. Publication No. 2005/02033143 or the crystalline Forms described in PCT/IB2019/056988. A single pharmaceutical composition may include two, three, four, or more than four different crystalline forms of eluxadoline. For example, a pharmaceutical composition may be composed of Forms C and C′; C and D′; C and H3; C and amorphous eluxadoline; C and α; or C and β. Similar combinations with other crystalline forms described in the instant application may also be composed.

The present invention is also directed to methods of isolating and preparing the crystal Forms C, C′, D′ or H3 of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid.

In some embodiments, the methods comprise first preparing Form A crystal of eluxadoline, which involve the steps of combining a strong ionizable acid eluxadoline to prepare a salt of eluxadoline; and washing said salt of eluxadoline with an inorganic base to obtain eluxadoline. In another embodiment, Form A crystals may be made in the process described in U.S. Pub. No. 2005/02033143, the contents of which are incorporated herein in their entirety. In some embodiments, the invention may further comprise the step of washing said eluxadoline with water. The inorganic base may be selected from sodium hydroxide, potassium hydroxide, sodium carbonate, sodium acetate, sodium phosphate. In some embodiments, the inorganic base is sodium hydroxide. The ionizable acid may be selected from hydrochloric acid, trifluoroacetic acid, formic acid, and phosphoric acid. In some embodiments, said ionizable acid is hydrochloric acid. In one embodiment, a method of preparing eluxadoline comprises the steps of: combining hydrochloric acid with eluxadoline to prepare the hydrochloride salt of eluxadoline; washing said salt of eluxadoline with sodium hydroxide; and washing said eluxadoline with water. The resulting eluxadoline is then added to dichloromethane and heated, the slurry stored at room temperature, the residue isolated and dried to prepare Form A crystal.

Form C may be obtained from adding one of methanol (MeOH), dichloromethane (DCM), tetrahydrofuran (THF) or tetrahydrofuran and water to Form A and recover Form C upon maturation followed by drying.

Form C′ may be formed the crystals growth when Form A is dissolved in methanol.

Form D′ may be formed from the dehydration of Form E (a hydrated form of eluxadoline). As described in PCT/IB2019/056988, Form A may be suspended in water and stirred for a period of time, with subsequent solids isolated, rinsed and dried to prepare Form D crystal. Form D may be stored in the presence of a drying agent to prepare Form E crystal. Subsequent dehydration of Form E results in Form D′.

Form H3 may be formed by slurrying Form A in water. Form H3 may convert back to Form A with some minor amorphous contents within some time, and completely convert to amorphous phase upon further slurrying within further time.

The crystals of the present invention were analyzed using the following methods.

### Powder X-Ray Diffraction

X-ray powder diffraction (XRPD) data was collected under ambient conditions by placing samples on a zero-background sample holder with a 0.1 mm indent. The data was generated by using a Rigaku Miniflex600 diffractometer with Cu K-α₁ (λ=1.5406 Angstrom) radiation at a diffraction angle range of 2 to 40° (20) with scan rate at 1 or 2° per minute and a step size of 0.01° (20), applying X-ray generator set up at 40 kV and 15 mA.

PDXL data analysis software for Rigaku Miniflex was used to generate diffractograms that present X-ray powder diffraction patterns of the polymorphs.

One of ordinary skill in the art will appreciate that a powder X-ray diffraction pattern may be obtained with a measurement error that is dependent upon the measurement conditions employed. In particular, it is generally known that intensities in a X-ray powder diffraction pattern may fluctuate depending upon measurement conditions employed. It should be further understood that relative intensities may also vary depending upon experimental conditions and, accordingly, the exact order of intensity should not be taken into account. Accordingly, the relative intensity of peaks in a diffractogram is not necessarily a limitation of the PXRD pattern because peak intensity can vary from sample to sample, e.g., due to crystalline impurities.

Additionally, a measurement error of diffraction angle for a conventional powder X-ray powder diffraction pattern is typically about 5% or less, and such degree of measurement error should be taken into account as pertaining to the aforementioned diffraction angles. Further, the angles of each peak can vary by about +/−0.1 degrees, or by about +/−0.05. The entire pattern or most of the pattern peaks may also shift by about +/−0.1 degrees to about +/−0.2 degrees due to differences in calibration, settings, and other variations from instrument to instrument. All reported XRPD peaks in the Figures, Examples, and elsewhere herein are reported with an error of about ±0.2 degrees 2-theta. Unless otherwise noted, all diffractograms are obtained at about room temperature (about 24 degrees C. to about 25 degrees C.). It is to be understood that the crystal structures of the instant invention are not limited to the crystal structures that provide X-ray diffraction patterns completely identical to the X-ray powder diffraction patterns depicted in the accompanying Figures disclosed herein. Any crystal structures that provide powder X-ray diffraction patterns substantially identical to those disclosed in the accompanying Figures fall within the scope of the present invention. The ability to ascertain substantial identities of X-ray powder diffraction patterns is within the purview of one of ordinary skill in the art.

### Single Crystal X-Ray Analysis (SCXR)

Single crystal X-ray data was generated by using Bruker D8 (QUEST) SCXR diffractometer, equipped with high brightness (IμS 3.0 microfocus) applying X-ray generator setting at 50 kV and 1 mA to obtain Cu Kα radiation (λ, =1.54 Å). PHOTON II Charge-Integrating Pixel Array Detector of superior speed, sensitivity, and accuracy was used for diffraction data collection on single crystals of Form C′ and Form D′.

Cryostream 800 low temperature device, which furnishes sample temperatures between 80 K and 500 K, was used to cool crystals at 173K (−100° C.). With the crystal in a cold nitrogen gas stream, reduced thermal motion of the atoms increases the crystal's scattering power leading to high quality structural data.

Bruker APEX3 software suite including SHELXTL was used to run diffraction experiments, data collections and integrations for refining the data and solving crystal structures.

### EXAMPLES

### Example 1: Preparation of the Form C Crystal of 5-({[2-Amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid

A 1 L three-necked round-bottomed flask equipped with a mechanical stirrer, addition funnel and a thermocouple was charged without agitation. 34.2 g of 5-({[2-tert-butoxycarbonylamino-3-(4-carbamoyl-2,6-dimethyl-phenyl)-propionyl]-[1-(4-phenyl-1H-imidazol-2-yl)-ethyl]-amino}-methyl)-2-methoxy-benzoic acid (see Example 9 of US 2005/0203143), 340 mL of acetone, and 17 mL of 204 mM concentrated HCl were combined in the flask. The stirring was started and the resulting slurry formed a clear solution. This solution was heated to 45° C. under vigorous stirring and aged at this temperature for a period of two hours. After the completion, the reaction mass was cooled to ambient temperature and the supernatant was removed by suction. The vessel along with the residue was rinsed with 20 ml of acetone and then removed as previously. 170 ml of water was added and the reaction mass and was aged under stirring until a homogeneous solution resulted. This solution was then added over a period of ˜½ hr to a solution of 90 ml of 1N NaOH and water. The pH was adjusted to 6.5-7.0 accordingly. The resulting slurry was aged for about 2 hrs at ambient temperature, cooled to 10-15° C., aged at that temperature for about 1 hr, and then filtered. The solid was washed with 10 ml water, air-dried for a period of 4 to 5 hrs, and then placed in a vacuum oven at 50-55° C. until the water content was less than 3%. The resulting Form A of 5-({[2-tert-butoxycarbonylamino-3-(4-carbamoyl-2,6-dimethyl-phenyl)-propionyl]-[1-(4-phenyl-1H-imidazol-2-yl)-ethyl]-amino}-methyl)-2-methoxy-benzoic acid was added to 3 ml dichloromethane and heated at 50° C. for 2 days. The slurry was stored at room temperature for 3 days. The residue was isolated and dried for 4 days at 40° C. to prepare Form A crystal.

Form C, a possible solvate, was obtained from three solvents, MeOH, DCM and THF, with difference in crystal quality observed. To identify and compare the solvents tested for the formation of Form C, 8 ml of each solvent (i.e. MeOH, DCM and THF) was added to 50 mg of the starting material Form A (JPZ732). The starting material dissolved faster in MeOH, appeared as a thin slurry, while DCM ad THF gave only partial dissolution and resulted a denser slurry instead. Form C was also identified in the solvent mixture of THF and water. Form C is an isostructural solvate of eluxadoline. Characteristic powder diffraction data of Form C is shown herein (XRPD pattern in FIG. 1).

### Example 2: Preparation of the Form C′ Crystal

Form C′ was prepared by dissolving Form A in methanol in a capped vial and after a few months, several thin block-like crystals were formed on the vial's wall. Form C′ is a tri-methanolate crystal of eluxadoline. The crystal structure of the single crystals was confirmed to be tri-methanolate. Characteristic powder diffraction data of Form C′ is shown herein (XRPD pattern in FIG. 2, simulated from SXCR data on Form C′). Single crystal structure and lattice parameters (a, b, c, α, β, γ, volume and density) for Form C′ are shown herein in FIG. 6, FIG. 7 and Table 1.

### Example 3: Preparation of the Form D′ Crystal

Form D′ was prepared upon dehydration of Form E. As further described in PCT/IB2019/056988, Form E was prepared by storing 1 gram of Form D in a desiccator containing Dririte as the drying agent for 3 days at ambient temperature. Form E is a partially dehydrated form of eluxadoline Form D. Form D was prepared by suspending 3 grams of Form A in 12 mL of water in a 20 mL vial. The contents of the vial were stirred for 9 days at 25° C. Solids were isolated by vacuum filtration, rinsed with water, and air dried. Form D′ is a monohydrate form of eluxadoline. Single crystals of Form D′ were obtained upon dehydration of Form E using crystal growth experiments. The SCXR analysis of the single crystals of Form D′ revealed that this new form is a monohydrate. Characteristic powder diffraction data of Form D′ is shown herein (XRPD pattern in FIG. 3).

### Example 4: Preparation of Form H3 Crystal (Trihydrate)

Form H3 was prepared by slurry of Form A in water upon stirring. Form H3 converted back to Form A with some minor amorphous content within 2 days, and completely converted to amorphous phase upon slurry within 8 days. After additional 7 days (i.e. day 15 of slurry experiments), the amorphous phase converted further to Form D (tetrahydrate) with high crystallinity. The slurry experiments confirmed that the commercial eluxadoline Form A converts to hydrated crystalline forms, i.e. trihydrate form (H3) and/tetrahydrate (Form D), when suspended in water.

Although the invention has been described with respect to various embodiments, it should be realized this invention is also capable of a wide variety of further and other embodiments within the spirit and scope of the appended claims.

What is claimed:

1. A pharmaceutical composition comprising a Form C crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, wherein the Form C crystal is characterized by a powder X-ray diffraction pattern having at least a minimum corresponding number of powder X-ray diffraction peaks selected from the group consisting of powder X-ray diffraction peaks at about 7.2±0.2, 11.8±0.2, 12.1±0.2, 12.7±0.2, 15.0±0.2, 15.8±0.2, 16.1±0.2, 18.2±0.2, 19.2±0.2, 19.9±0.2, 22.6±0.2, and 25.0±0.2 degrees 2-theta, wherein said minimum corresponding number is at least three.

2. The pharmaceutical composition of claim 1, wherein the Form C crystal is characterized by a powder X-ray diffraction pattern having any three or more powder X-ray diffraction peaks selected from the group consisting of powder X-ray diffraction peaks at about 7.2±0.2, 11.8±0.2, 12.1±0.2, 12.7±0.2, 15.0±0.2, 15.8±0.2, 16.1±0.2, 18.2±0.2, 19.2±0.2, 19.9±0.2, 22.6±0.2, and 25.0±0.2 degrees 2-theta.

3. The pharmaceutical composition of claim 1, wherein the Form C crystal is characterized by a powder X-ray diffraction pattern having powder X-ray diffraction peaks at about 7.2±0.2, 12.1±0.2 and 19.2±0.2 degrees 2-theta.

4. The pharmaceutical composition of claim 1, wherein the Form C crystal is characterized by a powder X-ray diffraction pattern having powder X-ray diffraction peaks at about 7.2±0.2, 12.1±0.2, 19.2±0.2 and 11.8±0.2 degrees 2-theta.

5. The pharmaceutical composition of claim 1, wherein the Form C crystal is characterized by a powder X-ray diffraction pattern having powder X-ray diffraction peaks at about 82±0.2, 12.1±0.2, 19.2±0.2, 11.8±0.2 and 15.0±0.2 degrees 2-theta.

6. The pharmaceutical composition of claim 1, wherein said minimum corresponding number is four.

7. The pharmaceutical composition of claim 1, wherein the Form C crystal is characterized by a powder X-ray diffraction pattern having powder X-ray diffraction peaks substantially similar to the powder X ray diffraction peaks of FIG. 1.

8. The pharmaceutical composition of claim 1, in a dosage form suitable for oral administration.

9. The pharmaceutical composition of claim 8, wherein the dosage form is a solid.

10. The pharmaceutical composition of claim 8, wherein the dosage form is selected from the group consisting of a tablet, a caplet, a hard gelatin capsule, a starch capsule, a hydroxypropyl methylcellulose (HPMC) capsule, and a soft elastic gelatin capsule.

11. The pharmaceutical composition of claim 8, wherein the dosage form as administered is a liquid.

12. The pharmaceutical composition of claim 8, wherein the dosage form as administered is selected from the group consisting of a suspension, a solution, a syrup, and an emulsion.

13. The pharmaceutical composition of claim 8, wherein the dosage form is a tablet.

14. A method of treating an opioid receptor disorder in a mammal comprising administering to the mammal an effective amount of the pharmaceutical composition of claim 1.

15. The method of claim 14, wherein the opioid receptor disorder is selected from the group consisting of irritable bowel syndrome, pain and a combination of both.

16. The method of claim 14, wherein the opioid receptor disorder is irritable bowel syndrome.

17. The method of claim 14, wherein the opioid receptor disorder is pain.

18. A pharmaceutical composition comprising a Form C′ crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, wherein the Form C′ crystal is characterized by a powder X-ray diffraction pattern having at least a minimum corresponding number of powder X-ray diffraction peaks selected from the group consisting of powder X-ray diffraction peaks at about 6.6±0.2, 6.7±0.2, 8.8±0.2, 10.4±0.2, 11.4±0.2, 11.8±0.2, 11.9±0.2, 13.2±0.2, 14.5±0.2, 17.6±0.2, 18.1±0.2, 20.2±0.2, 21.0±0.2, 21.7±0.2, 22.6±0.2, 23.2±0.2, 24.7±0.2 and 26.6±0.2 degrees 2-theta, wherein said minimum corresponding number is at least three.

19. The pharmaceutical composition of claim 18, wherein the Form C′ crystal is characterized by a powder X-ray diffraction pattern having any three or more powder X-ray diffraction peaks selected from the group consisting of powder X-ray diffraction peaks at about 6.6±0.2, 6.7±0.2, 8.8±0.2, 10.4±0.2, 11.4±0.2, 11.8±0.2, 11.9±0.2, 13.2±0.2, 14.5±0.2, 17.6±0.2, 18.1±0.2, 20.2±0.2, 21.0±0.2, 21.7±0.2, 22.6±0.2, 23.2±0.2, 24.7±0.2 and 26.6±0.2 degrees 2-theta.

20. The pharmaceutical composition of claim 18, wherein the Form C′ crystal is characterized by a powder X-ray diffraction pattern having powder X-ray diffraction peaks at about 6.7±0.2, 21.0±0.2 and 6.6±0.2 degrees 2-theta.

21. The pharmaceutical composition of claim 18, wherein the Form C′ crystal is characterized by a powder X-ray diffraction pattern having powder X-ray diffraction peaks at about 6.7±0.2, 21.0±0.2, 6.6±0.2 and 10.4±0.2 degrees 2-theta.

22. The pharmaceutical composition of claim 18, wherein the Form C′ crystal is characterized by a powder X-ray diffraction pattern having powder X-ray diffraction peaks at about 6.7±0.2, 21.0±0.2, 6.6±0.2, 10.4±0.2 and 11.8±0.2 degrees 2-theta.

23. The pharmaceutical composition of claim 18, wherein said minimum corresponding number is four.

24. The pharmaceutical composition of claim 18, wherein the Form C′ crystal is characterized by a powder X-ray diffraction pattern having powder X-ray diffraction peaks substantially similar to the powder X ray diffraction peaks of FIG. 2.

25. The pharmaceutical composition of claim 18, in a dosage form suitable for oral administration.

26. The pharmaceutical composition of claim 25, wherein the dosage form is a solid.

27. The pharmaceutical composition of claim 25, wherein the dosage form is selected from the group consisting of a tablet, a caplet, a hard gelatin capsule, a starch capsule, a hydroxypropyl methylcellulose (HPMC) capsule, and a soft elastic gelatin capsule.

28. The pharmaceutical composition of claim 25, wherein the dosage form as administered is a liquid.

29. The pharmaceutical composition of claim 25, wherein the dosage form as administered is selected from the group consisting of a suspension, a solution, a syrup, and an emulsion.

30. The pharmaceutical composition of claim 25, wherein the dosage form is a tablet.

31. A method of treating an opioid receptor disorder in a mammal comprising administering to the mammal an effective amount of the pharmaceutical composition of claim 18.

32. The method of claim 31, wherein the opioid receptor disorder is selected from the group consisting of irritable bowel syndrome, pain and a combination of both.

33. The method of claim 31, wherein the opioid receptor disorder is irritable bowel syndrome.

34. The method of claim 31, wherein the opioid receptor disorder is pain.

35. A pharmaceutical composition comprising a Form D′ crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, wherein the Form F crystal is characterized by a powder X-ray diffraction pattern having at least a minimum corresponding number of powder X-ray diffraction peaks selected from the group consisting of powder X-ray diffraction peaks at about 8.9±0.2, 9.2±0.2, 11.1±0.2, 11.3±0.2, 12.0±0.2, 13.7±0.2, 16.0±0.2, 17.4±0.2, 17.8±0.2, 17.9±0.2, 20.5±0.2, 20.8±0.2, 21.3±0.2, 21.7±0.2, 21.8±0.2, 22.0±0.2, 25.0±0.2, 26.8±0.2, 27.6±0.2 and 29.1±0.2 degrees 2-theta, wherein said minimum corresponding number is three.

36. The pharmaceutical composition of claim 35, wherein the Form D′ crystal is characterized by a powder X-ray diffraction pattern having any three or more powder X-ray diffraction peaks selected from the group consisting of powder X-ray diffraction peaks at about 8.9±0.2, 9.2±0.2, 11.1±0.2, 11.3±0.2, 12.0±0.2, 13.7±0.2, 16.0±0.2, 17.4±0.2, 17.8±0.2, 17.9±0.2, 20.5±0.2, 20.8±0.2, 21.3±0.2, 21.7±0.2, 21.8±0.2, 22.0±0.2, 25.0±0.2, 26.8±0.2, 27.6±0.2 and 29.1±0.2 degrees 2-theta.

37. The pharmaceutical composition of claim 35, wherein the Form D′ crystal is characterized by a powder X-ray diffraction pattern having powder X-ray diffraction peaks at about 16.0±0.2, 12.0±0.2 and 11.1±0.2 degrees 2-theta.

38. The pharmaceutical composition of claim 35, wherein the Form D′ crystal is characterized by a powder X-ray diffraction pattern having powder X-ray diffraction peaks at about 16.0±0.2, 12.0±0.2, 11.1±0.2 and 8.9±0.2 degrees 2-theta.

39. The pharmaceutical composition of claim 35, wherein the Form D′ crystal is characterized by a powder X-ray diffraction pattern having powder X-ray diffraction peaks at about 16.0±0.2, 12.0±0.2, 11.1±0.2, 8.9±0.2 and 27.6±0.2 degrees 2-theta.

40. The pharmaceutical composition of claim 35, wherein said minimum corresponding number is four.

41. The pharmaceutical composition of claim 35, wherein the Form D′ crystal is characterized by a powder X-ray diffraction pattern having powder X-ray diffraction peaks substantially similar to the powder X ray diffraction peaks of FIG. 3.

42. The pharmaceutical composition of claim 35, in a dosage form suitable for oral administration.

43. The pharmaceutical composition of claim 42, wherein the dosage form is a solid.

44. The pharmaceutical composition of claim 42, wherein the dosage form is selected from the group consisting of a tablet, a caplet, a hard gelatin capsule, a starch capsule, a hydroxypropyl methylcellulose (HPMC) capsule, and a soft elastic gelatin capsule.

45. The pharmaceutical composition of claim 42, wherein the dosage form as administered is a liquid.

46. The pharmaceutical composition of claim 42, wherein the dosage form as administered is selected from the group consisting of a suspension, a solution, a syrup, and an emulsion.

47. The pharmaceutical composition of claim 42, wherein the dosage form is a tablet.

48. A method of treating an opioid receptor disorder in a mammal comprising administering to the mammal an effective amount of the pharmaceutical composition of claim 35.

49. The method of claim 48, wherein the opioid receptor disorder is selected from the group consisting of irritable bowel syndrome, pain and a combination of both.

50. The method of claim 48, wherein the opioid receptor disorder is irritable bowel syndrome.

51. The method of claim 48, wherein the opioid receptor disorder is pain.

52. A pharmaceutical composition comprising a Form H3 crystal of 5-({[2-amino-3-(4-carbamoyl-2,6-dimethyl-phenyl)-propionyl]-[1-(4-phenyl-1H-imidazol-2-yl)-ethyl]-amino}-methyl)-2-methoxy-benzoic acid, wherein the Form F crystal is characterized by a powder X-ray diffraction pattern having at least a minimum corresponding number of powder X-ray diffraction peaks selected from the group consisting of powder X-ray diffraction peaks at about 8.1±0.2, 11.0±0.2, 12.4±0.2, 13.2±0.2, 14.8±0.2, 15.2±0.2, 16.6±0.2, 17.9±0.2, 18.7±0.2, 18.9±0.2, 19.1±0.2, 20.0±0.2 and 24.3±0.2 degrees 2-theta, wherein said minimum corresponding number is three.

53. The pharmaceutical composition of claim 52, wherein the Form H3 crystal is characterized by a powder X-ray diffraction pattern having any three or more powder X-ray diffraction peaks selected from the group consisting of powder X-ray diffraction peaks at about 8.1±0.2, 11.0±0.2, 12.4±0.2, 13.2±0.2, 14.8±0.2, 15.2±0.2, 16.6±0.2, 17.9±0.2, 18.7±0.2, 18.9±0.2, 19.1±0.2, 20.0±0.2 and 24.3±0.2 degrees 2-theta.

54. The pharmaceutical composition of claim 52, wherein the Form H3 crystal is characterized by a powder X-ray diffraction pattern having powder X-ray diffraction peaks at about 8.1±0.2, 14.8±0.2 and 16.6±0.2 degrees 2-theta.

55. The pharmaceutical composition of claim 52, wherein the Form H3 crystal is characterized by a powder X-ray diffraction pattern having powder X-ray diffraction peaks at about 8.1±0.2, 14.8±0.2, 16.6±0.2 and 17.9±0.2 degrees 2-theta.

56. The pharmaceutical composition of claim 52, wherein the Form H3 crystal is characterized by a powder X-ray diffraction pattern having powder X-ray diffraction peaks at about 8.1±0.2, 14.8±0.2, 16.6±0.2, 17.9±0.2 and 19.1±0.2 degrees 2-theta.

57. The pharmaceutical composition of claim 52, wherein said minimum corresponding number is four.

58. The pharmaceutical composition of claim 52, wherein the Form H3 crystal is characterized by a powder X-ray diffraction pattern having powder X-ray diffraction peaks substantially similar to the powder X ray diffraction peaks of FIG. 4.

59. The pharmaceutical composition of claim 52, in a dosage form suitable for oral administration.

60. The pharmaceutical composition of claim 52, wherein the dosage form is a solid.

61. The pharmaceutical composition of claim 59, wherein the dosage form is selected from the group consisting of a tablet, a caplet, a hard gelatin capsule, a starch capsule, a hydroxypropyl methylcellulose (HPMC) capsule, and a soft elastic gelatin capsule.

62. The pharmaceutical composition of claim 59, wherein the dosage form as administered is a liquid.

63. The pharmaceutical composition of claim 59, wherein the dosage form as administered is selected from the group consisting of a suspension, a solution, a syrup, and an emulsion.

64. The pharmaceutical composition of claim 59, wherein the dosage form is a tablet.

65. A method of treating an opioid receptor disorder in a mammal comprising administering to the mammal an effective amount of the pharmaceutical composition of claim 52.

66. The method of claim 65, wherein the opioid receptor disorder is selected from the group consisting of irritable bowel syndrome, pain and a combination of both.

67. The method of claim 65, wherein the opioid receptor disorder is irritable bowel syndrome.

68. The method of claim 65, wherein the opioid receptor disorder is pain.
